(19) United States
(12) Patent Application Publication
(10) Pub. No.: US 2019/0029742 A1
(43) Pub. Date: Jan. 31, 2019

(54) DEVICES, SYSTEMS, AND METHODS FOR ACETABULAR FRACTURE FIXATION USING A LIMITED OPEN SURGICAL APPROACH

(71) Applicant: BOARD OF REGENTS, THE UNIVERSITY OF TEXAS SYSTEM, Austin, TX (US)

(72) Inventors: Philip C. Jarrett, Dallas, TX (US); Emiliya G. Usheva, Dallas, TX (US); Brendan G. Swain, Houston, TX (US); Brigham K. Au, Coppell, TX (US)

(21) Appl. No.: 16/045,931

(22) Filed: Jul. 26, 2018

Related U.S. Application Data
(60) Provisional application No. 62/538,458, filed on Jul. 28, 2017.

(51) Int. Cl. A61B 17/80; A61B 17/17

(52) U.S. Cl. CPC A61B 17/8066; A61B 2017/564; A61B 17/1728; A61B 17/1746

(57) ABSTRACT

The present invention includes a device, system, and method to repair acetabulum fractures that can include a surgical plate, guide arm, alignment arm, and a locking mechanism to permit the fixation of acetabulum fractures involving the quadrilateral surface of the acetabulum. This invention may significantly increase the strength of fixation by applying compression external to the bony matrix.

### CROSS-REFERENCE TO RELATED APPLICATIONS

This patent application claims priority to U.S. provisional patent application Ser. No. 62/538,458 filed on Jul. 28, 2017 entitled “Devices, Systems, and Methods for Pelvic Stabilization Using a Limited Open Surgical Approach,” all of which is hereby incorporated by reference in its entirety.

### TECHNICAL FIELD OF THE INVENTION

The present invention relates generally to a device, system and method to repair acetabulum fractures and stabilize the pelvis by limited open surgery. More specifically, the present invention utilizes compression that prevents medial displacement of acetabular fracture components independent of the bone strength.

### STATEMENT OF FEDERALLY FUNDED RESEARCH

Not applicable.

### INCORPORATION-BY-REFERENCE OF MATERIALS FILED ON COMPACT DISC

Not applicable.

### BACKGROUND OF THE INVENTION

Without limiting the scope of the invention, its background is described in connection with orthopedic surgery for pelvic fractures.

There are a variety of circumstances under which a patient with an acetabular fracture may need fracture fixation. For example, elderly individuals represent the most rapidly growing subgroup of patients sustaining acetabular fractures, and the incidence rate of these fractures is expected to double over the next twenty years. Additionally, patients suffering from osteoporosis are at a significantly higher risk for fractures. In the year 2000, there were an estimated 9 million osteoporotic fractures, of which 1.6 million were at the hip. The current methods include open reduction internal fixation, in which surgery is needed to realign the bone fracture into a normal position. Internal fixation refers to the rods, screws, or plates used to keep the bone fracture stable in order to facilitate the healing process. Drawbacks to this surgery include damage to the muscle and bone, nerve damage, movement of plates, screws, and pins, and possible future surgeries to remove the internal fixation. Current solutions to high-risk elderly, osteoporotic, or pelvic ring patients use one or more plates positioned on the medial surface of the quadrilateral plate that receive medial-to-lateral screws. Acetabular fracture fixation and stabilization depends on the inherent mechanical properties of the bone, namely, the fragile nature of osteoporotic bone as the patient walks and mobilizes, thus leaving the fixation susceptible to failure.

U.S. Patent Application Publication No. 2012/0010617 (herein after the '617 patent application) describes a system for inserting a surgical fixation plate medial to the ilium and ischium for fractures of the acetabulum. A guide system is used to position the plate in the true pelvis on the medial aspect of the quadrilateral surface, and this system uses a cannulated drill guide external to the body that directs a drill bit through infra-acetabular bone from lateral to medial. The drilled bone allows direct access through the bone to one aperture of the inserted plate for a screw or bolt. A screw or bolt inserted through the drilled aperture allows for plate and fracture compression that prevents mediolateral displacement of the fractured components common in multiple acetabular fracture patterns. The compression of the plate and fracture is not dependent on the bone mineral density, allowing for patients with severe osteoporosis to achieve surgical reduction and fixation uncompromised by bone strength. However, this application has significant limitations and drawbacks. A significant problem with drilling or surgical manipulation of the infra-acetabular region is the risk of damage to the sciatic nerve where the patient may sustain lifelong neuropathy, paresthesia, or limb paralysis. Another drawback of the method in this application is that the cannulated guide fails to provide a method to drill into additional apertures of the plate without compromising the acetabulum. To apply compression across a fracture, rigid fasteners such as screws or bolts must be applied on both fracture components. The '617 patent application achieves such compression with the anterior fracture component but does not provide for compression in the posterior fracture component that achieves such compression without relying on bone strength. In addition, it uses a bolt that latches onto the plate on the medial end and receives a screw on the lateral end. These combinations of metal components achieve compression but metal-to-metal contact can generate microscopic metal debris in the patient during movement from shear wear-and-tear. The surgical approach described in the art possesses multiple weaknesses that are addressed by the current patent application. Thus, a need remains for improved devices, systems, and methods for the repair of acetabulum fractures.

### SUMMARY OF THE INVENTION

In one embodiment, the present invention includes acetabulum stabilization device comprising: a surgical plate comprising one or more surgical plate apertures and a guide arm tab locating slot positioned in an edge of the surgical plate; a guide arm comprising a locking connector and a guide plate end connected by a connector section to a lateral plate guide, a guide arm tab positioned on the surgical plate end is capable of mating with the guide arm tab locating slot, wherein the lateral guide plate comprises one or more lateral guide plate apertures, wherein the guide arm is capable of releasably attaching to the surgical plate; and a drill guide comprising a longitudinal rod, wherein the longitudinal rod is adapted to be fitted removably through the lateral guide plate apertures, wherein the drill guide is capable of directing a drill to drill holes in a bone about an acetabular fracture. In one aspect, the guide arm further comprises an alignment arm attached to the guide arm capable of releasably attaching to the surgical plate at the connector aperture with a locking connector. In another aspect, the surgical plate, the guide arm, alignment arm, cannulated drill guide, buttons, fasteners and rope comprise at least one of surgical steel, polymer, titanium, metals, alloys, or any material with suitable malleability and rigidity. In another aspect, the device further comprises one or more notches on the surgical plate to fit to the bone of a patient. In another aspect, the device further comprises one or more notches on a right side, a bottom side or a left side of the surgical plate. In another aspect, the device further comprises an alignment locking pin on the guide arm configured to secure the alignment arm to the alignment guide. In another aspect, the device further comprises an alignment locking pin on the alignment arm configured to secure the alignment arm to the alignment guide. In another aspect, the locking connector slides through the surgical plate connector and guide arm locking hole to lock the surgical plate, the alignment arm, and the guide arm into one rigid assembly. In another aspect, the locking connector slides through the surgical plate connector and guide arm locking hole to fixate the surgical plate, the alignment arm, and the guide arm into one rigid assembly.

In another embodiment, the present invention includes a kit for acetabulum stabilization comprising: a surgical plate comprising one or more surgical plate apertures and a guide arm tab locating slot positioned in an edge of the surgical plate; a guide arm comprising a locking connector and a guide plate end connected by a connector section to a lateral plate guide, a guide arm tab positioned on the surgical plate end is capable of mating with the guide arm tab locating slot, wherein the lateral guide plate comprises one or more lateral guide plate apertures, wherein the guide arm is capable of releasably attaching to the surgical plate; and a drill guide comprising a longitudinal rod, wherein the longitudinal rod is adapted to be fitted removably through the lateral guide plate apertures, wherein the drill guide is capable of directing a drill to the drill holes in a bone about a fracture in the acetabulum fracture; and a set of fasteners wherein each fastener comprises a set of two or more fastener apertures capable of attaching the surgical plate to the pelvic bone. In one aspect, the guide arm further comprises an alignment arm attached to the guide arm capable of releasably attaching to the surgical plate at the connector aperture with a locking connector. In another aspect, the kit further comprises one or more of the notches on the surgical plate to fit to the bone of a patient. In another aspect, the kit further comprises one or more of the notches on a right side, a bottom side, and or a left side, or on any other desired sides of the surgical plate.

In another embodiment, the present invention includes a method of repairing an acetabulum fracture of a pelvis, the method comprising the steps of: providing an acetabulum stabilization device comprising: a surgical plate comprising one or more surgical plate apertures and a guide arm tab locating slot positioned in an edge of the surgical plate; a guide arm comprising a locking connector and a guide plate end connected by a connector section to a lateral plate guide, a guide arm tab positioned on the surgical plate end is capable of mating with the guide arm tab locating slot, wherein the lateral guide plate comprises one or more lateral guide plate apertures, wherein the guide arm is capable of releasably attaching to the surgical plate; positioning the surgical plate against an interior surface of the pelvis; positioning the lateral guide plate against an iliac crest of the pelvis to achieve an optimal drilling angle; locking the guide arm to immobilize the surgical plate; inserting the cannulated drill guide through a first lateral guide plate aperture to align with the corresponding first surgical plate aperture of the set of surgical plate apertures; drilling one or more bone apertures through the cannulated drill guide through the pelvis in a direction from a lateral surface of the pelvis toward an interior of the pelvis; and fastening the surgical plate to the pelvis. In one aspect, the guide arm further comprises an alignment arm attached to the guide arm capable of releasably attaching to the surgical plate at the connector aperture with a locking connector. In another aspect, the method further comprises the step of unlocking the surgical plate from the guide arm. In another aspect, the method further comprises the step of removing the guide arm and closing the patient. In another aspect, the surgical plate further comprises one or more notches to create a customized fit to the patient. In another aspect, the surgical plate comprises at least one of stainless steel, polymer composite, titanium, or any other suitable material or a combination thereof. In another aspect, the method further comprises the step of adjusting the suture to compress and stabilize a fracture. In another aspect, suture comprises at least one or surgical steel, stainless steel, titanium, polypropylene, polyester, silk, nylon or a combination thereof.

In yet another embodiment, the present invention includes an acetabulum stabilization device for the repair of acetabulum fractures, wherein the device comprises: a surgical plate comprising: a surgical plate front and a surgical plate back separated by a surgical plate edge, a set of surgical plate apertures that penetrates from the surgical plate front to the surgical plate back and a guide arm tab locating slot positioned in the surgical plate edge; and a surgical plate connector aperture that penetrates from the surgical plate front to the surgical plate back and through the guide arm tab locating slot; a guide arm comprising: a surgical plate end and a guide plate end connected by a connector section; and a guide arm tab positioned on the surgical plate end that mates with the guide arm tab locating slot; a lateral guide plate attached to the guide plate end wherein the lateral guide plate comprises a lateral guide plate front opposite a lateral guide plate back with a set of lateral guide plate apertures that penetrate from the lateral guide plate front to the lateral guide plate back; and an alignment guide attached to the connector section and comprising an alignment arm aperture; an alignment arm comprising: a medial end and a lateral end connected by an alignment arm connector section wherein the alignment arm connector section slidably mates to an alignment arm aperture and a male locking connector positioned at the medial end to frictionally fit the surgical plate connector aperture; a cannulated drill guide comprising a longitudinal rod extending away from a head, wherein the longitudinal rod is adapted to be fitted removably through the lateral guide plate aperture; a set of buttons wherein each button comprises a set of button apertures that extend from a button front to a button back; a set of fasteners wherein each fastener comprises a set of two or more fastener apertures that extend from a fastener front to a fastener back; and a suture with a diameter less than the diameter of the surgical plate apertures and fastener apertures.

In yet another embodiment, the present invention includes a method of repairing an acetabulum fracture, the method comprising the steps of: providing an acetabulum stabilization device comprising: a surgical plate comprising: a surgical plate front and a surgical plate back separated by a surgical plate edge, a set of surgical plate apertures that penetrates from the surgical plate front to the surgical plate back and a guide arm tab locating slot positioned in the surgical plate edge; and a surgical plate connector aperture that penetrates from the surgical plate front to the surgical plate back and through the guide arm tab locating slot, a guide arm comprising: a surgical plate end and a guide plate end connected by a connector section; and a guide arm tab positioned on the surgical plate end removably positioned in the guide arm tab locating slot; a lateral guide plate attached to the guide plate end wherein the lateral guide plate comprises a lateral guide plate front opposite a lateral guide plate back with a set of lateral guide plate apertures that penetrate from the lateral guide plate front to the lateral guide plate back; an alignment guide attached to the connector section and comprising an alignment arm aperture; and an alignment arm comprising: a medial end and a lateral end connected by an alignment arm connector section wherein the alignment arm connector section is slidably mated to the alignment arm aperture and a male locking connector; positioned at the medial end is frictionally fitted in the surgical plate connector aperture; positioning the surgical plate against an interior surface of the pelvis; positioning the lateral guide plate against an iliac crest of the pelvis to achieve an optimal drilling angle; locking the guide arm to alignment arm to immobilize the surgical plate; inserting the cannulated drill guide through a first lateral guide plate aperture of the set of lateral guide plate apertures to align with the corresponding first surgical plate aperture of the set of surgical plate apertures; drilling one or more bone apertures through the cannulated drill guide through the pelvis in a direction from a lateral surface of the pelvis toward the medial or quadrilateral surface of the pelvis; placing a set of buttons in contact with the surgical plate; placing a set of fasteners on the medial side of the pelvis opposite the corresponding set of buttons; and securing the set of buttons to the set of fasteners through the one or more bone apertures using a suture to form a compression.

The surgical plate can be unlocked from the guide arm and the alignment arm, and the acetabulum stabilization device can be removed from the pelvis, and the patient can be closed. The surgical plate may be further composed of one or more notch(es) to create a customized fit to the bone of a patient, and the surgical plate can be composed of stainless steel, polymer composite, titanium, or any other suitable material. The suture may be adjusted to compress and stabilize a fracture, and this suture may be made of a composite of surgical steel or other surgical non-absorbable material such as stainless steel, titanium, polypropylene, polyester, silk, and or nylon.

### BRIEF DESCRIPTION OF THE DRAWINGS

For a more complete understanding of the features and advantages of the present invention, reference is now made to the detailed description of the invention along with the accompanying figures and in which:

FIG. 1A is a cross section of the femoral joint.

FIG. 1B is an image showing osteoporotic and normal bone.

FIG. 2A is an image showing an anterior view of the pelvis.

FIG. 2B is an image showing a lateral view of the pelvis.

FIGS. 2C through 2H are lateral views of the pelvis show showing various acetabular fractures.

FIG. 3 is a perspective view of the surgical plate including five surgical plate apertures, three fasteners, and suture.

FIG. 4 is a perspective view of the buttons and button apertures that are tied with sutures to the bone and fasteners through the fastener apertures.

FIG. 5 is a perspective view of the current device before the surgical plate, guide arm, and alignment arm lock into place.

FIG. 6 is a perspective view of the current device after the surgical plate, guide arm, and alignment arm lock into place.

FIG. 7 is a perspective view of the current device after the surgical plate, guide arm, and alignment arm lock into place. Additionally, this illustration shows the cannulated drill guide positioned through a lateral guide plate aperture in anticipation of drilling.

FIG. 8 is a perspective view of one embodiment of the current device in a model pelvic girdle with the guide arm and alignment arm locked to the surgical plate.

FIG. 9 is a lateral view of one embodiment of the current device in a model pelvic girdle with the guide arm and alignment arm locked to the surgical plate.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. It is contemplated that any embodiment discussed in this specification can be implemented with respect to any method, kit, reagent, or composition of the invention, and vice versa. Furthermore, compositions of the invention can be used to achieve methods of the invention.

The present invention relates generally to a method, composition, and system to repair acetabulum fractures and stabilize the pelvis by limited open surgery. More specifically, the present invention utilizes compression with suture, which prevents mediolateral displacement of acetabular fracture components independent of the bone strength. The suture may be adjusted to compress and stabilize a fracture, and this suture may be made of a composite of surgical steel or other surgical non-absorbable material such as stainless steel, titanium, polypropylene, polyester, silk, and or nylon.

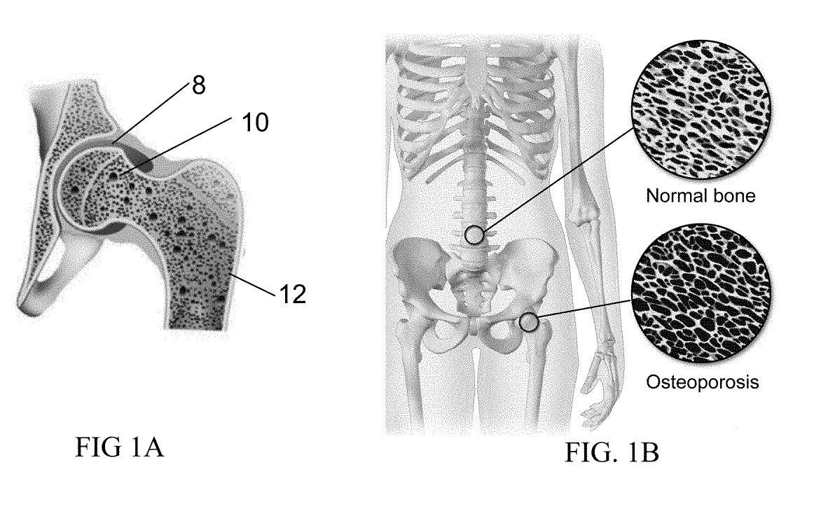

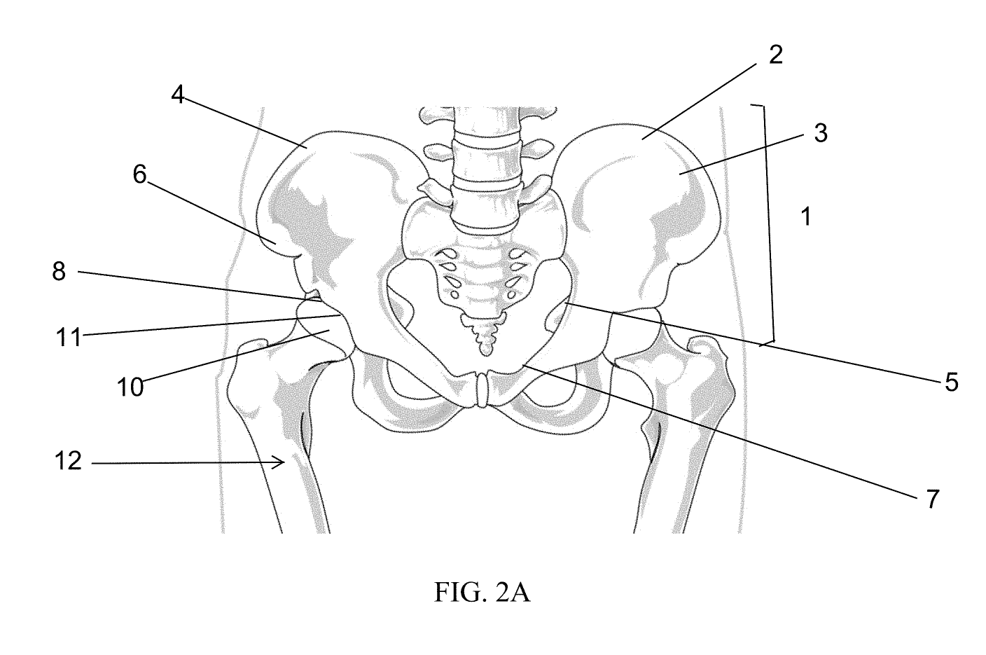

FIG. 1A is a cross sectional image showing an osteoporotic femoral joint with the femoral head 10 extending from the femur 12 positioned into the acetabulum 8. The acetabulum 8 contains a concave surface in which the femoral head 10 is located to form the hip joint. FIG. 1B is an image showing a close-up of osteoporotic bone with a visualization of decreased bone mass and density and an image showing the density of normal bone. Fractures of the acetabulum may arise from a variety of issues including trauma, osteoporosis, old age, etc., and these fractures tend to be difficult to repair due to the complex configuration and location of the acetabulum, and the abundance of soft tissue structures such as nerves and vessels. Current methods to acetabulum repair include large incisions, and potential injury to the sciatic nerve. In contrast, the present invention discloses embodiments that repair acetabulum injuries using less invasive surgery with the use of a smaller incision and provide external compression and fixation with a plate contoured to the iliac crest.

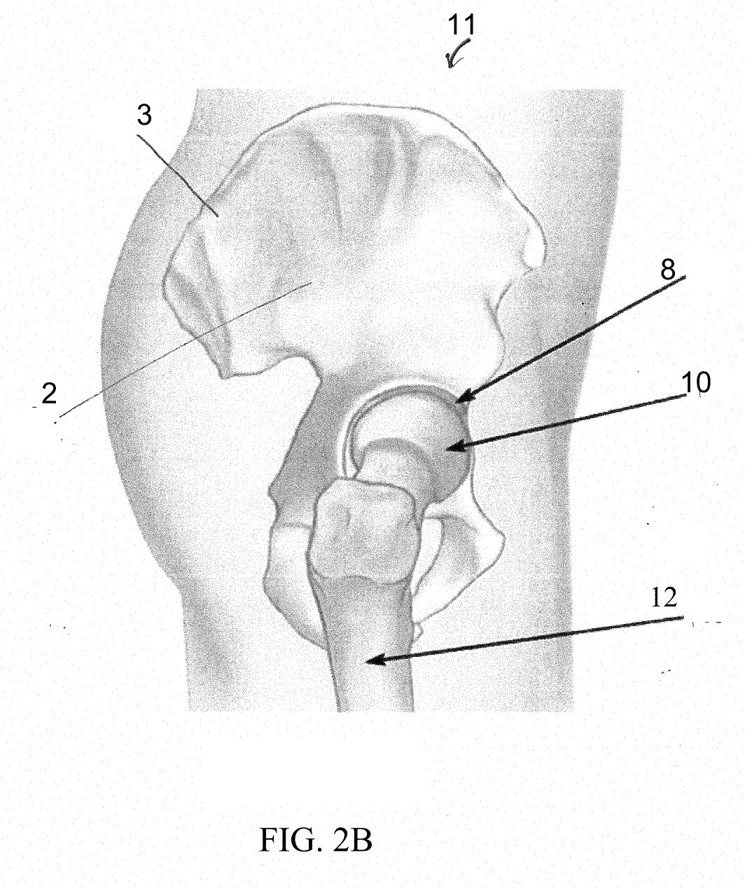

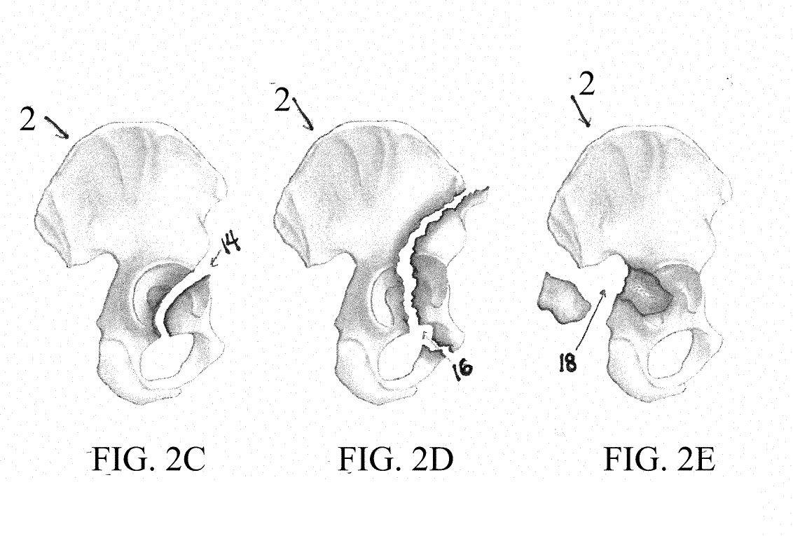

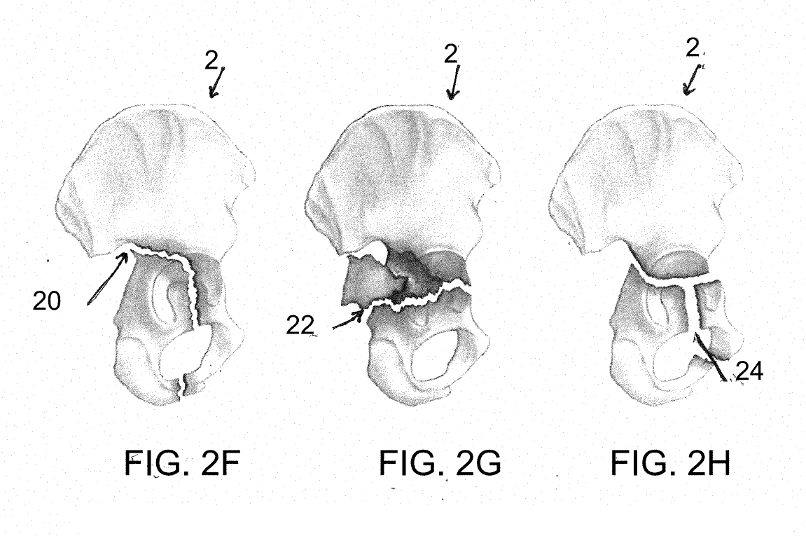

FIGS. 2A to 2H are images that show the various acetabulum fracture patterns of the pelvis. FIG. 2A is image showing an anterior view of the pelvic girdle 1 with the pelvis 2. The pelvis 2 houses the ilium 3, which is the largest portion of the superior part of the acetabulum 8. Anteriorly, the ilium 3 has an anterior superior iliac spine 6. From the anterior superior iliac spine 6, anteriorly, the iliac crest 4 forms laterally. FIG. 2A also shows the hip joint 11, which is a ball and socket synovial joint. The ball is the femoral head 10, and the socket is the acetabulum 8. The hip joint 11 is the articulation of the pelvis 2 with the femur 12. FIG. 2B is an image showing lateral view of the hip joint 11. This hip joint 11 is formed by the femoral head 10 of the femur 12 inserting into the acetabulum 8. The decreased density and strength of osteoporotic bone lends itself to more acetabulum fractures like the ones shown in FIGS. 2C-2H. FIGS. 2C-2H illustrate the pelvis 2 with common acetabular fractures 14, 16, 18, 20, 22, and 24.

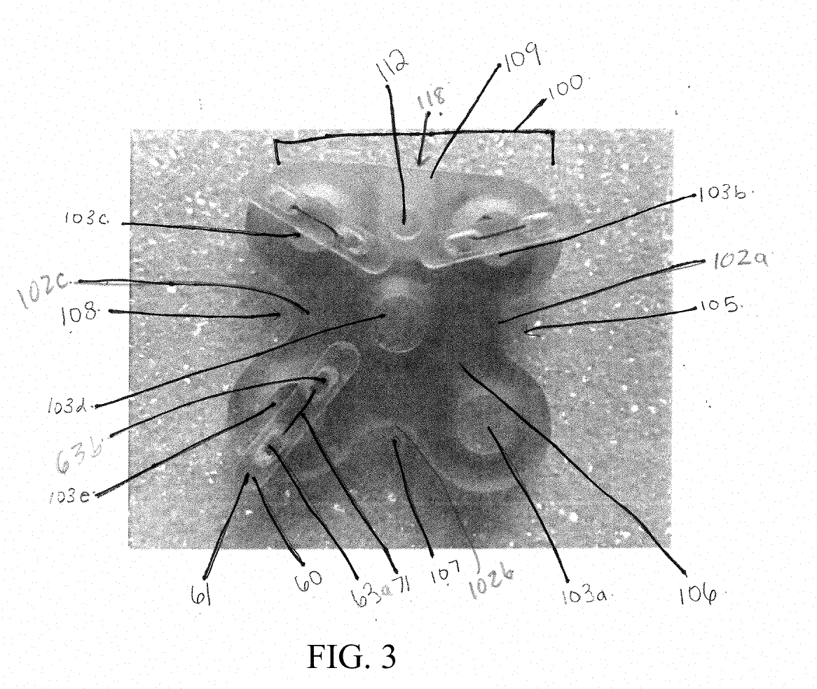

FIG. 3 is an image showing a top view of a surgical plate 100 for use with the present invention with surgical plate apertures 103a, 103b, 103c, 103d and 103e, which can be fastened to the bone via fasteners 60. The surgical plate 100 may be made of, e.g., surgical steel, or any other surgical non-absorbable material such as stainless steel, titanium, polypropylene, polyester, silk, and/or nylon, or any desired material deemed appropriate. The surgical plate 100 is substantially planar and has a medial side 106 and a lateral side 115 (not shown in this figure). The medial side 106 comes into contact with the bone through suture compression. The surgical plate 100 includes a top side 109, and a guide arm tab locating aperture 118, which is located in the center of the top side 109. Between the medial side 106 and the lateral side 115, the surgical plate 100 can also include one or more notches 102a, 102b, and 102c to accommodate bone surface. In the illustrated embodiment, the notch 102a is on the right side 105, notch 102b is on the bottom side 107, and notch 102c is on the left side 108. These notches 102, 102b, 102c are optional, and can be included on any side of the surgical plate 100. The one or more surgical plate apertures 103a, 103b, 103c, 103d, and 103e can also have a countersunk hole or edge that extends from, e.g., the medial side 106 to the lateral side (not shown). The number and shape of the surgical plate apertures 103a-103e can be varied based on the desired characteristics of the surgical plate 100. The illustrated surgical plate 100 contains five surgical plate apertures 103a-103e but more or fewer openings may be desired. In one embodiment, these illustrated surgical plate apertures 103a-103e are circular in shape with each surgical plate aperture being symmetrical and identical to one another. In one embodiment, these illustrated surgical plate apertures 103a-103e can also be circular, oval, or any other geometrical or free form shape with a countersink feature in each surgical plate aperture 103a-103e where the larger opening diameter is on the medial side 106 of the surgical plate 100 and the narrower diameter size is on the lateral side 115 of the surgical plate 100. While inventive aspects may lie in the measurements used herein, other measurements and ranges may be used in other embodiments. The surgical plate 100 also contains a surgical plate connector aperture 112 that runs from the medial side 106 of the surgical plate through to the opposite side. This surgical plate connector aperture 112 passes through the guide arm tab locating aperture 118 in order to lock the device. In the illustrated embodiment, the surgical plate connector aperture 112 is located proximally below the top side 109 and the guide arm tab locating aperture 118. FIG. 3 also illustrates the mechanism behind suture compression. This illustration shows the surgical plate 100 secured to buttons (not shown) with sutures 71 and fasteners 60 creating a compression effect by tying the buttons (not shown) and the pelvic girdle (not shown) to the surgical plate 100. In order to achieve compression, one or more sutures 71 are threaded from the lateral side of the bone through the fastener apertures 63a, 63b of the fastener 60 and through the button apertures (not shown) of the buttons (not shown) aligned with the surgical plate apertures 103a-103e.

The fasteners 60 may have any shape desired provided that it spans a greater diameter than the surgical plate apertures 103 and buttons (not shown). The fasteners 60 can be made of surgical steel, polymer, titanium or any desired material to achieve desired malleability and/or rigidity and have any shape, such as round, oval, square, rectangular, or polygonal. The fasteners 60 can include two or more fastener apertures 63a and 63b that span from fastener front 61 to back side (not shown). The number and shape of the fastener apertures 63a-63b can be selected based on the desired characteristics of the fastener. The illustrated embodiment includes two fastener apertures 63a and 63b, but more fastener apertures may be desirable. While inventive aspects may lie in the measurements used herein, other measurements and ranges may be used in other embodiments.

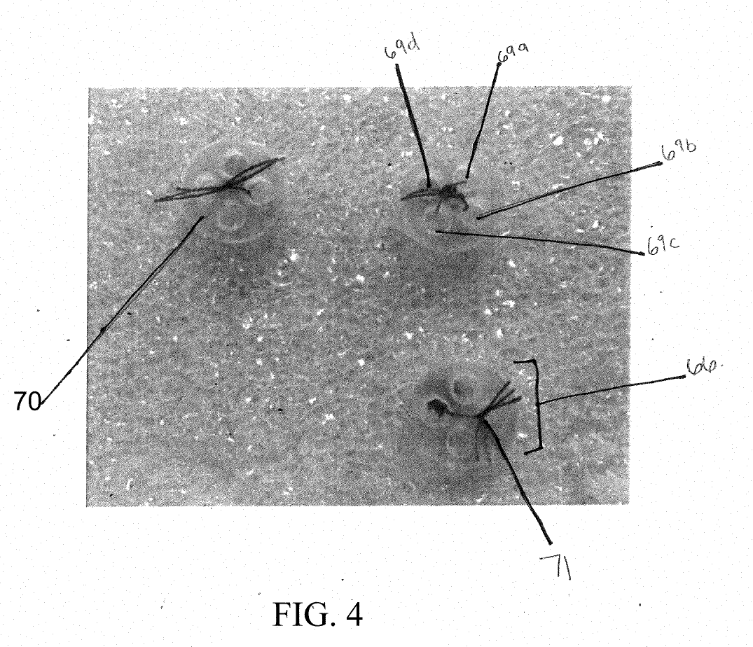

FIG. 4 shows images of buttons 66 that are generally circularly planar and include button apertures 69a, 69b 69c, and 69d, and are shown with a suture 71. The surgical plate 100 (not shown) is secured by the buttons 66 to create a tight compression. The buttons 66 can be made of surgical steel, titanium, polymer composite, and/or nylon or any desired material to achieve desired malleability and/or rigidity. The buttons 66 contain two or more button apertures 69a-69d which are apertures running through the body of the button 66 from the front side 70 to back side (not shown) of the button. In this illustrated embodiment, the buttons 66 are identical and symmetrical but the number and shape of the apertures(s) can be selected based on the desired characteristics of the button 66. The illustrated embodiment includes four button apertures 69a, 69b, 69c, and 69d, but more or fewer button apertures may be used. While inventive aspects may lie in the measurements used herein, other measurements and ranges may be used in other embodiments.

The suture 71 is used to tie together the buttons 66 through button apertures 69a-69d into openings in the bone. The suture 71 may be composed of a composite of surgical steel or other surgical non-absorbable material such as stainless steel, titanium, polypropylene, polyester, silk, and or nylon. The suture 71 can extend from button 66 through an aperture in the bone (not shown) the surgical plate apertures (not shown) into fastener 60 to secure the surgical plate 100 to the bone (not shown). The suture 71 can be adjusted to secure the surgical plate 100 to the bone (not shown) to compress the fracture.

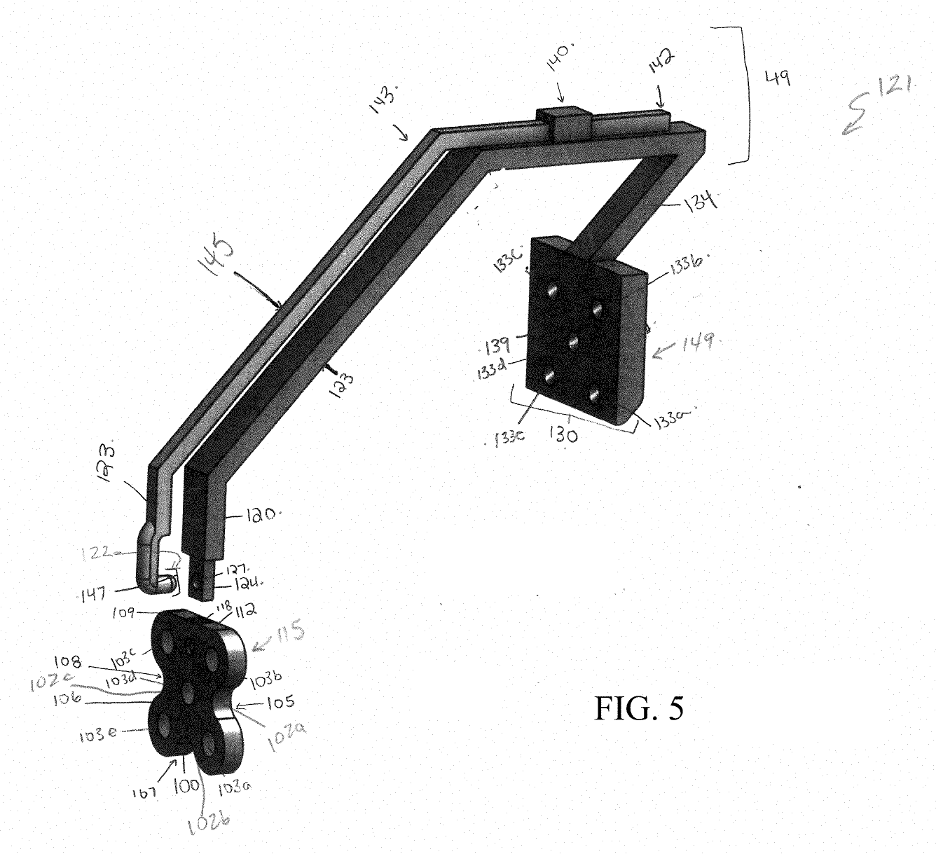

FIG. 5 is one embodiment of the dual arm system 49 and surgical plate 100 prior to assembly. The image shows the surgical plate 100 with the surgical plate apertures 103a, 103b, 103c, 103d, and 103e. The surgical plate 100 is substantially planar and has a medial side 106 and a lateral side 115. The lateral side 115 comes into contact with the bone (not shown) through suture compression. The surgical plate 100 includes a top side 109, and a guide arm tab locating aperture 118 which is located in the center of the top side 109. Between the medial side 106 and the lateral side 115, the surgical plate 100 can also include one or more notches 102a, 102b, and 102c to accommodate bone surface. In the illustrated embodiment, the notch 102a is on the right side 105, notch 102b is on the bottom side 107, and notch 102c is on the left side 108. These notches 102a-102c can be included on any side of the surgical plate 100. The surgical plate 100 can further include one or more surgical plate apertures 103a, 103b, 103c, 103d, and 103e that extend from the medial side 106 to the lateral side 115. The number and shape of the surgical plate apertures 103a-103e can be varied based on the desired characteristics of the surgical plate 100. The illustrated surgical plate contains five surgical plate apertures 103a-103e but more or fewer surgical plate apertures may be desired. In one embodiment, these illustrated surgical plate apertures 103a-103e are circular in shape with each surgical plate aperture being symmetrical and identical to one another. In one embodiment, these illustrated surgical plate apertures 103a-103e are circular, oval, or any other geometrical or free form shape with a countersink feature in each surgical plate aperture 103 where the larger opening diameter is on the medial side 106 of the surgical plate 100 and the narrower diameter size is on the lateral side 115 of the surgical plate. The surgical plate 100 also contains a surgical plate connector aperture 112 that runs from the medial side 106 of the surgical plate 100 to the lateral side 115. This surgical plate connector aperture 112 passes through the guide arm tab locating aperture 118 in order to lock the device. In the illustrated embodiment, the surgical plate connector aperture 112 is located proximally below the top side 109 and the guide arm tab locating aperture 118. While inventive aspects may lie in the measurements used herein, other measurements and ranges may be used in other embodiments.

FIG. 5 also shows guide arm 121 having a surgical plate attachment end 120 and a guide plate attachment end 134 connected by connector end 123. This guide arm 121 may be composed of any desired material such as surgical steel, titanium, a polymer composite, etc. In this embodiment, the guide arm 121 is rigidly attached to a lateral guide plate 130 on the guide plate attachment end 134. On surgical plate attachment end 120, the guide arm 121 has a guide arm tab 124 with a guide arm tab-locking aperture 127 extending through the guide arm tab 124. This guide arm tab-locking aperture 127 is placed in the guide arm tab 124 of the guide arm 121. An alignment guide 140 that is rigidly fixed to the guide arm 121 slidably engages an alignment arm 143.

Running parallel to the guide arm 121 is the alignment arm 143 that slidably engages with the alignment guide 140. This longitudinal alignment arm 143 is able to translate relative to the fixed guide arm 121, and may be composed of any desired material such as surgical steel, titanium, a polymer composite, etc. The alignment arm 143 is configured to move along a linear path when translating relative to the guide arm 121 and through the passageway of the alignment guide 140 attached to the guide arm 121. In the illustrated embodiments, the guide arm 121 and alignment arm 143 have a substantially rectangular outer perimeter along the longitudinal length thereof. This alignment arm 143 includes a protruding male locking connector 147 on its lateral end 122.

The guide arm 121 includes attached alignment guide 140 that cooperates with the alignment arm 143 to permit movement relative to the guide arm 121. The alignment arm 143 includes an end 142 (that may also include a handle or other biasing mechanism to provide for motion and return to an initiation position after releasably locking the alignment arm 143 to the surgical plate 100), a middle section 154, and connector end 123. In other embodiments that will be illustrated in further figures, this alignment arm 143 translates to allow the guide arm 121 and alignment arm 143 to properly align and mate with the surgical plate 100. In FIGS. 5-7, the alignment guide 140 can define a substantially rectangular inner perimeter that is complementary to and can slide over the outer perimeter of the alignment arm 143. Other desired arrangements or embodiments for achieving translational and non-rotational motions between the alignment guide 140 and the alignment arm 143 are possible. In other embodiments, complementarities other then the substantially rectangular inner and outer perimeters may be used as deemed fit.

Importantly, while FIG. 5 shows an embodiment of a dual arm system 49, however, the skilled artisan will recognize that the present invention may use a single arm (e.g., guide arm 121) that includes a reliable locking mechanism at surgical plate attachment end 120 that include an internal releasable locking mechanism, e.g., using guide wires, notches, springs, or other methods for releasable locking the guide arm 121 to the surgical plate.

FIG. 5 also illustrates a lateral guide plate 130 that is rigidly attached to and located on the guide plate attachment end 134 of the guide arm 121. This lateral guide plate 130 can be crafted from surgical steel, titanium, polymer composite, or any material deemed fit. One embodiment of the current device can include a lateral guide plate 130 that is substantially planar, and may be rectangular in shape but other arrangements or complementarities are possible. The lateral guide plate 130 can further include a medial side 139 of the lateral guide plate 130 and a lateral side 149 of the lateral guide plate 130. Additionally, the lateral guide plate 130 can include one or more apertures each entitled a lateral guide plate aperture(s) 133a, 133b, 133c, 133d, and 133e that run from the medial side 139 of the lateral guide plate 130 to the lateral side 149 of the lateral guide plate 130. The number and shape of the lateral guide plate aperture(s) 133a-133e can be selected based on the desired characteristics of the lateral guide plate 130. The illustrated lateral guide plate 130 contains five identical circular lateral guide plate apertures or openings of different sizes. Other embodiments may include different locking mechanism such as a male connector on the surgical plate 100 and or a female counterpart on any of the alignment arms 143 or guide arms 121. While inventive aspects may lie in the measurements used herein, other measurements and ranges may be used in other embodiments.

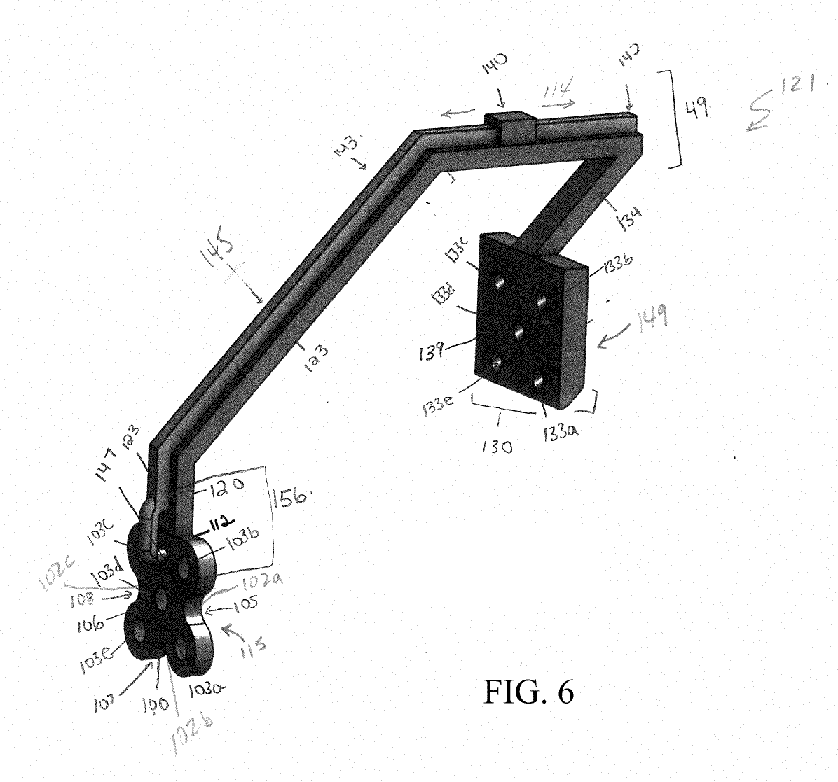

FIGS. 6-9 show the operation of the dual arm system 49 with the guide arm 121, surgical plate 100, and the alignment arm 143 in a locked position at 156. In reference to FIG. 6, for the locked position 156 of the guide arm 121 and surgical plate 100 to occur, the male connector engages the surgical plate 100 via sliding 114, in which the male connector inserts into the surgical plate connector aperture 112 and mates to the guide arm tab 124 of the guide arm 121.

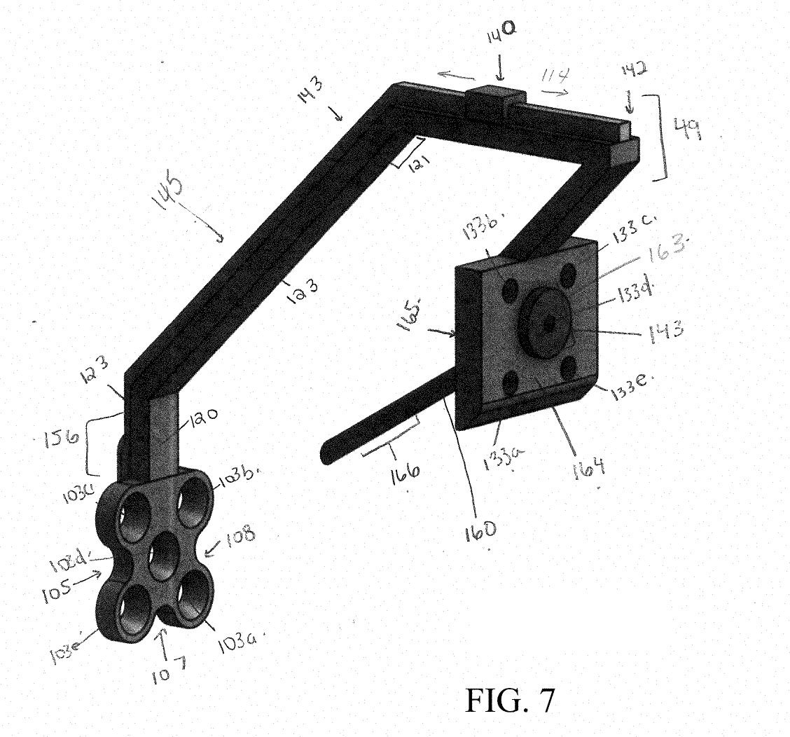

FIG. 7 shows an image of an embodiment of the device where the surgeon uses a cannulated drill guide 160 as a drilling guide to anticipate or direct the path of drilling. In this embodiment, the device is in the locked position 156. The cannulated drill guide 160 is a substantially cylindrical object with a nail-like shape, containing two rigidly connected parts. On the lateral end 165 of the cannulated drill guide 160, there is a flat circular head 163. On the medial end 164 of the cannulated drill guide 160, there is an elongated longitudinal rod 166 that inserts into one of the lateral guide plate apertures 133a, 133b, 133c, 133d, and 133e. The cannulated drill guide 160 may be composed of any material deemed fit for cannulated drills.

Once the assembly is complete and the cannulated drill guide 160 is positioned through a lateral guide plate aperture 133a-133e, the surgeon makes an incision less than 1 cm in length down the acetabular bone (not shown). The surgeon then drills through the bone in the ventral to dorsal direction into the surgical plate apertures 103a-103e. The surgeon repeats this drilling process until 3-5 apertures are drilled. Next, sutures (not shown) are passed through each surgical plate apertures 103a-103e and used to generate compression on the medial side 106 of the surgical plate 100. The end of each sutures (not shown) is fastened to a button 66 (not shown) and a fastener 60 (not shown) through the button apertures 69a-69d (not shown) and fastener apertures (not shown) to apply lateral compression similar to a screw head. While sutures are depicted, any type of fastener can be used in its place. This suture compression provides a stable anchoring of the bone to the surgical plate 100. The various steps of or stages of the addition of additional buttons (not shown) can follow the steps above with respect to the first button (not shown) and fastener (not shown) pair. Once all the desired buttons (not shown) and fasteners (not shown) have been attached via the suture (not shown) to the surgical plate 100, the surgical plate 100 can be unmated and unlocked from the guide arm 121. After, the guide arm 121 is removed, and the patient (not shown) is closed in any suitable manner.

By fixing the surgical plate 100 to the pelvic girdle (not shown) with buttons (not shown) and fasteners (not shown), compression of the fracture (not shown) is achieved as the surgical plate 100 is placed against the fracture (not shown), itself. Depending on the location of the fracture (not shown) or any other fracture, the surgical plate 100 may be positioned distal to the acetabulum 8 (not shown) and dorsal to the acetabulum 8 within the pelvis 2 (not shown). Some embodiments of the surgical plate 100 can push the quadrilateral plate of the acetabulum 8 back to its original position. During stabilization and compression or after it has been achieved, an image intensifier can be used to confirm fixation and compression as desired.

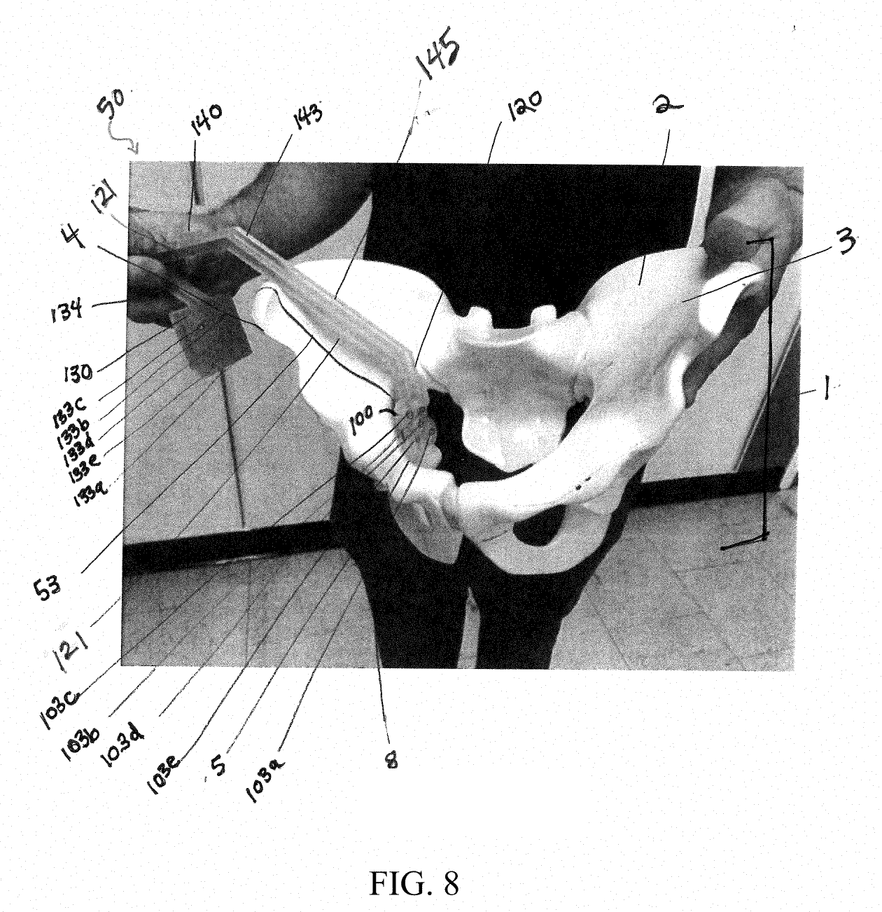

FIG. 8 is an image of the current device inserted into the pelvic girdle 1. In this illustration, the rigid assembly 50 is inserted into the pelvic girdle 1, with the surgical plate 100 resting within the pelvis 2 on the quadrilateral plate of the acetabulum 8 and on the medial portion of the ilium 3 proximal to the pelvic brim 5. The guide arm 121 and alignment arm 143 are positioned parallel to the fracture 53, and lateromedially along the interior surface of the ilium 3 the interior surface of the ilium 3, and the lateral guide plate 130 is externally lateral to the iliac crest 4.

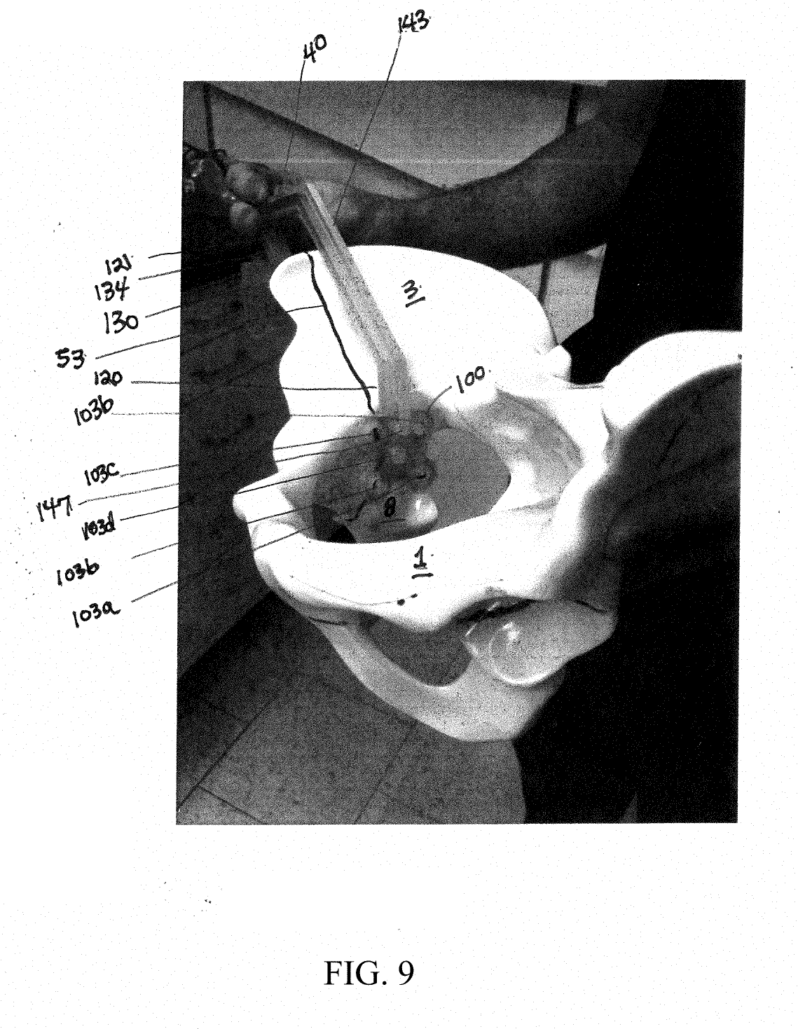

FIG. 9 is an image that illustrates a lateral view of the rigid assembly inserted into the pelvic girdle 1 with the surgical plate 100 resting on the interior of the quadrilateral plate of the acetabulum 8. The guide arm 121 and alignment arm 143 are sitting parallel to fracture 53 and alignment arm 143 along the ilium 3. The guide arm 121 is positioned lateromedially along the interior surface of the ilium 3.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An acetabulum stabilization device comprising:
a surgical plate comprising one or more surgical plate apertures and a guide arm tab locating slot positioned in an edge of the surgical plate;
a guide arm comprising a locking connector and a guide plate end connected by a connector section to a lateral plate guide, a guide arm tab positioned on the surgical plate end is capable of mating with the guide arm tab locating slot, wherein the lateral guide plate comprises one or more lateral guide plate apertures, wherein the guide arm is capable of releasably attaching to the surgical plate; and
a drill guide comprising a longitudinal rod, wherein the longitudinal rod is adapted to be fitted removably through the lateral guide plate apertures, wherein the drill guide is capable of directing a drill to drill holes in a bone about an acetabular fracture.

2. The device from claim 1, wherein the guide arm further comprises an alignment arm attached to the guide arm capable of releasably attaching to the surgical plate at the connector aperture with a locking connector.

3. The device from claim 1, wherein the surgical plate, the guide arm, alignment arm, cannulated drill guide, buttons, fasteners and rope comprise at least one of surgical steel, polymer, titanium, metals, alloys, or any material with suitable malleability and rigidity.

4. The device from claim 1, further comprising one or more notches on the surgical plate to fit to the bone of a patient.

5. The device from claim 1, further comprising one or more notches on a right side, a bottom side or a left side of the surgical plate.

6. The device from claim 1, further comprising an alignment locking pin on the guide arm configured to secure the alignment arm to the alignment guide.

7. The device from claim 2, further comprising an alignment locking pin on the alignment arm configured to secure the alignment arm to the alignment guide.

8. The device from claim 2, wherein the locking connector slides through the surgical plate connector and guide arm locking hole to lock the surgical plate, the alignment arm, and the guide arm into one rigid assembly.

9. The device from claim 2, wherein the locking connector slides through the surgical plate connector and guide arm locking hole to fixate the surgical plate, the alignment arm, and the guide arm into one rigid assembly.

10. A kit for acetabulum stabilization comprising:
a surgical plate comprising one or more surgical plate apertures and a guide arm tab locating slot positioned in an edge of the surgical plate;
a guide arm comprising a locking connector and a guide plate end connected by a connector section to a lateral plate guide, a guide arm tab positioned on the surgical plate end is capable of mating with the guide arm tab locating slot, wherein the lateral guide plate comprises one or more lateral guide plate apertures, wherein the guide arm is capable of releasably attaching to the surgical plate; and
a drill guide comprising a longitudinal rod, wherein the longitudinal rod is adapted to be fitted removably through the lateral guide plate apertures, wherein the drill guide is capable of directing a drill to the drill holes in a bone about a fracture in the acetabulum fracture; and
a set of fasteners wherein each fastener comprises a set of two or more fastener apertures capable of attaching the surgical plate to the pelvic bone.

11. The kit from claim 10, wherein the guide arm further comprises an alignment arm attached to the guide arm capable of releasably attaching to the surgical plate at the connector aperture with a locking connector.

12. The kit from claim 10, further comprising one or more of the notches on the surgical plate to fit to the bone of a patient.

13. The kit from claim 10, further comprising one or more of the notches on a right side, a bottom side, and or a left side, or on any other desired sides of the surgical plate.

14. A method of repairing an acetabulum fracture of a pelvis, the method comprising the steps of:
providing an acetabulum stabilization device comprising:
a surgical plate comprising one or more surgical plate apertures and a guide arm tab locating slot positioned in an edge of the surgical plate;
a guide arm comprising a locking connector and a guide plate end connected by a connector section to a lateral plate guide, a guide arm tab positioned on the surgical plate end is capable of mating with the guide arm tab locating slot, wherein the lateral guide plate comprises one or more lateral guide plate apertures, wherein the guide arm is capable of releasably attaching to the surgical plate;
positioning the surgical plate against an interior surface of the pelvis;
positioning the lateral guide plate against an iliac crest of the pelvis to achieve an optimal drilling angle;
locking the guide arm to immobilize the surgical plate;
inserting the cannulated drill guide through a first lateral guide plate aperture to align with the corresponding first surgical plate aperture of the set of surgical plate apertures;
drilling one or more bone apertures through the cannulated drill guide through the pelvis in a direction from a lateral surface of the pelvis toward an interior of the pelvis; and
fastening the surgical plate to the pelvis.

15. The method of claim 14, wherein the guide arm further comprises an alignment arm attached to the guide arm capable of releasably attaching to the surgical plate at the connector aperture with a locking connector.

16. The method of claim 14, further comprising the step of unlocking the surgical plate from the guide arm.

17. The method of claim 14, further comprising the step of removing the guide arm and closing the patient.

18. The method of claim 14, wherein the surgical plate further comprises one or more notches to create a customized fit to the patient.

19. The method of claim 14, wherein the surgical plate comprises at least one of stainless steel, polymer composite, titanium, or any other suitable material or a combination thereof.

20. The method of claim 14, further comprising the step of adjusting the suture to compress and stabilize a fracture.

21. The method of claim 14, wherein the suture comprises at least one or surgical steel, stainless steel, titanium, polypropylene, polyester, silk, nylon or a combination thereof.

22. An acetabulum stabilization device for the repair of acetabulum fractures, wherein the device comprises:
a surgical plate comprising:
a surgical plate front and a surgical plate back separated by a surgical plate edge,
a set of surgical plate apertures that penetrates from the surgical plate front to the surgical plate back and a guide arm tab locating slot positioned in the surgical plate edge; and
a surgical plate connector aperture that penetrates from the surgical plate front to the surgical plate back and through the guide arm tab locating slot;
a guide arm comprising:
a surgical plate end and a guide plate end connected by a connector section; and
a guide arm tab positioned on the surgical plate end that mates with the guide arm tab locating slot;
a lateral guide plate attached to the guide plate end wherein the lateral guide plate comprises a lateral guide plate front opposite a lateral guide plate back with a set of lateral guide plate apertures that penetrate from the lateral guide plate front to the lateral guide plate back; and
an alignment guide attached to the connector section and comprising an alignment arm aperture;
an alignment arm comprising:
a medial end and a lateral end connected by an alignment arm connector section wherein the alignment arm connector section slidably mates to an alignment arm aperture and a male locking connector positioned at the medial end to frictionally fit the surgical plate connector aperture;
a cannulated drill guide comprising a longitudinal rod extending away from a head, wherein the longitudinal rod is adapted to be fitted removably through the lateral guide plate aperture;
a set of buttons wherein each button comprises a set of button apertures that extend from a button front to a button back;
a set of fasteners wherein each fastener comprises a set of two or more fastener apertures that extend from a fastener front to a fastener back; and
a suture with a diameter less than the diameter of the surgical plate apertures and fastener apertures.

23. A method of repairing an acetabulum fracture, the method comprising the steps of:
providing an acetabulum stabilization device comprising:
a surgical plate comprising:
a surgical plate front and a surgical plate back separated by a surgical plate edge,
a set of surgical plate apertures that penetrates from the surgical plate front to the surgical plate back and a guide arm tab locating slot positioned in the surgical plate edge; and
a surgical plate connector aperture that penetrates from the surgical plate front to the surgical plate back and through the guide arm tab locating slot,
a guide arm comprising:
a surgical plate end and a guide plate end connected by a connector section; and
a guide arm tab positioned on the surgical plate end removably positioned in the guide arm tab locating slot;
a lateral guide plate attached to the guide plate end wherein the lateral guide plate comprises a lateral guide plate front opposite a lateral guide plate back with a set of lateral guide plate apertures that penetrate from the lateral guide plate front to the lateral guide plate back;
an alignment guide attached to the connector section and comprising an alignment arm aperture; and
an alignment arm comprising:
a medial end and a lateral end connected by an alignment arm connector section wherein the alignment arm connector section is slidably mated to the alignment arm aperture and a male locking connector; positioned at the medial end is frictionally fitted in the surgical plate connector aperture;
positioning the surgical plate against an interior surface of the pelvis;
positioning the lateral guide plate against an iliac crest of the pelvis to achieve an optimal drilling angle;
locking the guide arm to alignment arm to immobilize the surgical plate;
inserting the cannulated drill guide through a first lateral guide plate aperture of the set of lateral guide plate apertures to align with the corresponding first surgical plate aperture of the set of surgical plate apertures;
drilling one or more bone apertures through the cannulated drill guide through the pelvis in a direction from a lateral surface of the pelvis toward the medial or quadrilateral surface of the pelvis;
placing a set of buttons in contact with the surgical plate;
placing a set of fasteners on the medial side of the pelvis opposite the corresponding set of buttons; and
securing the set of buttons to the set of fasteners through the one or more bone apertures using a suture to form a compression.
